(19) United States
(12) Patent Application Publication
(10) Pub. No.: US 2010/0324139 A1
(43) Pub. Date: Dec. 23, 2010

(54) PROCESS TO PREGABALIN

(75) Inventors: Abhay Gaitonde, Maharashtra (IN); Debashish Datta, Maharashtra (IN); Bindu Manojkumar, Maharashtra (IN); Sunanda Phadtare, Maharashtra (IN)

Correspondence Address: FULBRIGHT & JAWORSKI L.L.P.;Attn: MN IP Docket, 600 Congress Avenue, Suite 2400, Austin, TX, 78701, US

(73) Assignees: Generics [UK] Limited, Hertfordshire (GB); Mylan India Private Limted, Maharashtra (IN)

(21) Appl. No.: 12/810,244

(22) PCT Filed: Dec. 19, 2008
(86) PCT No.: PCT/GB2008/051221

§ 371 Date: Sep. 10, 2010

(30) Foreign Application Priority Data
Dec. 26, 2007 (IN) 1729/KOL/2007

(51) Int. Cl. C07C 227/04; C07C 201/10; C07C 227/34; C07C 69/716; C07C 69/675; C07C 69/63; C07C 229/08; A61K 31/197; A61P 29/00; A61P 9/10; A61P 25/24; A61P 25/22; A61P 25/08; A61P 25/18

(52) U.S. Cl. 514/561; 560/156; 562/553; 562/401; 560/174; 560/179; 560/226

(57) ABSTRACT

The present invention relates to a novel method for the preparation of racemic pregabalin (1) or a single enantiomer thereof, (S)-(+)-3-(aminomethyl)-5-methyl-hexanoic acid (2).

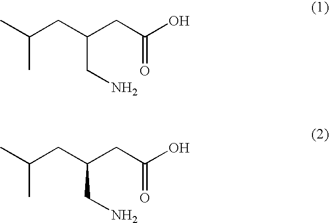

### CROSS-REFERENCE TO RELATED APPLICATION(s)

This application is a Section 371 National Stage Application of International No. PCT/GB2008/051221, filed 19 Dec. 2008 and published as WO 2009/081208 A1 on 2 Jul. 2009, which claims priority from the IN Patent Application No. 1729/KOL/2007, filed 26 Dec. 2007, the contents of which are incorporated herein in their entirety for all purposes.

### FIELD OF THE INVENTION

The present invention relates to a novel method for the preparation of racemic pregabalin (1) or a single enantiomer thereof, (S)-(+)-3-(aminomethyl)-5-methyl-hexanoic acid (2).

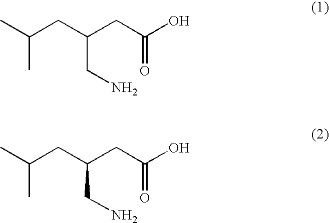

### BACKGROUND OF THE INVENTION

Pregabalin, (S)-(+)-3-aminomethyl-5-methyl-hexanoic acid (2), is related to the endogenous inhibitory neurotransmitter gamma-aminobutyric acid (GABA), which is involved in the regulation of brain neuronal activity. Pregabalin exhibits anti-seizure activity and is also thought to be useful for treating, amongst other conditions, pain, physiological conditions associated with psychomotor stimulants, inflammation, gastrointestinal damage, alcoholism, insomnia, fibromyalgia and various psychiatric disorders, including mania and bipolar disorder.

Racemic pregabalin was first reported in Synthesis, 1989, 953. The synthetic process reported involved the addition of nitromethane to an ethyl 2-alkenoate and the nitro ester thus formed was reduced using palladium on carbon. Subsequent hydrolysis using hydrochloric acid afforded racemic pregabalin as the hydrochloride salt. The free base of racemic pregabalin was prepared by ion exchange chromatography.

An alternative process, reported in U.S. Pat. No. 5,637,767, describes the condensation of isovaleraldehyde with diethyl malonate. The 2-carboxy-2-alkenoic acid thus formed is then reacted with a cyanide source, specifically potassium cyanide, and the subsequent product is hydrolyzed using KOH to give the potassium salt of the cyano acid which is hydrogenated in-situ using sponge nickel and neutralized with acetic acid to give racemic pregabalin.

An alternative process for the preparation of racemic pregabalin hydrochloride has been reported in US 2005/0043565. This process involves a Horner modification of a Wittig reaction between isovaleraldehyde and triethyl phosphonoacetate to afford the ethyl 2-alkenoate. Addition of nitromethane followed by hydrogenation using Raney nickel affords the lactam, which is hydrolyzed using hydrochloric acid to form the hydrochloride salt of the amino acid. The route reported in US 2005/0043565 gives the hydrochloride salt instead of the free base and it is well known that there are practical difficulties in the isolation of amino acids from aqueous media, due to the formation of zwitterionic species. The formation of the HCl salt of racemic pregabalin necessitates an aqueous work-up, which generally leads to poor yields and lengthy work-up procedures.

The present inventors were interested in preparing racemic pregabalin (1) and its single (S)-enantiomer (2) by the most convenient and shortest route. The route should also avoid the use of hazardous and environmentally unsuitable reagents (e.g. highly toxic KCN or potentially hazardous sponge nickel) and have simpler and more efficient work-up procedures than the known processes.

Preparation of pregabalin (2) can be achieved by following any of the processes described above for the preparation of racemic pregabalin (1) and including the additional step(s) of a classical resolution of a racemic intermediate or of the final product. However, resolution of pregabalin (1) itself leads to the loss of 50% of the racemic material and there is no reported method for recovery of the unwanted (R)-isomer.

The above limitations can be overcome by asymmetric synthesis of pregabalin. However, as explained below, the processes reported in the prior art for the asymmetric synthesis of pregabalin (2) are not very efficient or convenient for commercial manufacture.

The process disclosed in EP 1250311 utilises the reaction of isobutyraldehyde and acrylonitrile to afford 3-hydroxy-4-methyl-2-methylenepentanenitrile, which is converted in a number of steps to ethyl 3-cyano-5-methyl-hex-3-enoate. Asymmetric reduction of this compound using the proprietary ligand catalyst [(R,R)-MeDuPHOS]Rh(COD)]⁺BF₄⁻ in the presence of hydrogen gas followed by salt breaking affords pregabalin (2). However, this synthesis appears to be technologically very complex and, in addition, bisphosphine ligands, including the above proprietary ligand catalyst, are often difficult to prepare, which adds to their cost.

The process disclosed in EP 641330 utilises expensive chiral auxiliaries and organometallic chemistry which is expensive and potentially hazardous and, in this case, affords modest yields and purity.

Therefore there is a need for an efficient, simple and non-hazardous process for the preparation of enantiomerically pure pregabalin (2), which can optionally be used as an efficient alternative method for the preparation of racemic pregabalin (1).

### DEFINITIONS

For the purposes of the present invention, an “alkyl” group is defined as a monovalent saturated hydrocarbon, which may be straight-chained or branched, or be or include cyclic groups. An alkyl group may optionally be substituted, and may optionally include one or more heteroatoms N, O or S in its carbon skeleton. Preferably an alkyl group is straight-chained or branched. Preferably an alkyl group is not substituted. Preferably an alkyl group does not include any heteroatoms in its carbon skeleton. Examples of alkyl groups are methyl, ethyl, n-propyl, i-propyl, n-butyl, i-butyl, sec-butyl, t-butyl, n-pentyl, cyclopentyl, n-hexyl, cyclohexyl, n-heptyl and cycloheptyl groups. Preferably an alkyl group is a C₁₋₁₂ alkyl group, preferably a C₁₋₆ alkyl group. Preferably a cyclic alkyl group is a C₃₋₁₂ cyclic alkyl group, preferably a C₅₋₇ cyclic alkyl group.

An “alkenyl” group is defined as a monovalent hydrocarbon, which comprises at least one carbon-carbon double bond, which may be straight-chained or branched, or be or include cyclic groups. An alkenyl group may optionally be substituted, and may optionally include one or more heteroatoms N, O or S in its carbon skeleton. Preferably an alkenyl group is straight-chained or branched. Preferably an alkenyl group is not substituted. Preferably an alkenyl group does not include any heteroatoms in its carbon skeleton. Examples of alkenyl groups are vinyl, allyl, but-1-enyl, but-2-enyl, cyclohexenyl and cycloheptenyl groups. Preferably an alkenyl group is a C₂₋₁₂ alkenyl group, preferably a C₂₋₆ alkenyl group. Preferably a cyclic alkenyl group is a C₃₋₁₂ cyclic alkenyl group, preferably a C₅₋₇ cyclic alkenyl group.

An “alkynyl” group is defined as a monovalent hydrocarbon, which comprises at least one carbon-carbon triple bond, which may be straight-chained or branched, or be or include cyclic groups. An alkynyl group may optionally be substituted, and may optionally include one or more heteroatoms N, O or S in its carbon skeleton. Preferably an alkynyl group is straight-chained or branched. Preferably an alkynyl group is not substituted. Preferably an alkynyl group does not include any heteroatoms in its carbon skeleton. Examples of alkynyl groups are ethynyl, propargyl, but-1-ynyl and but-2-ynyl groups. Preferably an alkynyl group is a C₂₋₁₂ alkynyl group, preferably a C₂₋₆ alkynyl group.

An “aryl” group is defined as a monovalent aromatic hydrocarbon. An aryl group may optionally be substituted, and may optionally include one or more heteroatoms N, O or S in its carbon skeleton. Preferably an aryl group is not substituted. Preferably an aryl group does not include any heteroatoms in its carbon skeleton. Examples of aryl groups are phenyl, naphthyl, anthracenyl and phenanthrenyl groups. Preferably an aryl group is a C₄-C₁₄ aryl group, preferably a C₆-C₁₀ aryl group.

For the purposes of the present invention, where a combination of groups is referred to as one moiety, for example, arylalkyl, arylalkenyl, arylalkynyl, alkylaryl, alkenylaryl or alkynylaryl, the last mentioned group contains the atom by which the moiety is attached to the rest of the molecule. A typical example of an arylalkyl group is benzyl.

An “alkoxy” group is defined as a —O-alkyl, —O-alkenyl, —O-alkynyl, —O-aryl, —O-arylalkyl, —O-arylalkenyl, —O-arylalkynyl, —O-alkylaryl, —O-alkenylaryl or —O-alkynylaryl group. Preferably an “alkoxy” group is a —O-alkyl or —O-aryl group. More preferably an “alkoxy” group is a —O-alkyl group.

An “acyl” group is defined as a —CO-alkyl, —CO-alkenyl, —CO-alkynyl, —CO-aryl, —CO-arylalkyl, —CO-arylalkenyl, —CO-arylalkynyl, —CO-alkylaryl, —CO-alkenylaryl or —CO-alkynylaryl group. Preferably an “acyl” group is a —CO-alkyl or —CO-aryl group. More preferably an “acyl” group is a —CO-alkyl group.

A “silyl” group is defined as a —SiR^{y}₃ group, wherein each R^{y} is independently selected from an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton. Preferably a “silyl” group is a trimethylsilyl (TMS), triethylsilyl, triisopropylsilyl, dimethylisopropylsilyl, diethylisopropylsilyl, dimethyl-t-hexylsilyl, t-butyldimethylsilyl (TBDMS), t-butyldiphenylsilyl (TBDPS), tribenzylsilyl, tri-p-xylylsilyl, triphenylsilyl (TPS), diphenylmethylsilyl (DPMS), or t-butylmethoxyphenylsilyl (TBMPS) group.

A “halo” group is a fluoro, chloro, bromo or iodo group.

A “hydroxy” group is a —OH group. A “nitro” group is a —NO₂ group. An “amino” group is a —NH₂ group. A “carboxy” group is a —CO₂H group.

For the purposes of this invention, an optionally substituted alkyl, alkenyl, alkynyl, aryl, arylalkyl, arylalkenyl, arylalkynyl, alkylaryl, alkenylaryl or alkynylaryl group may be substituted with one or more of —F, —Cl, —Br, —I, —CF₃, —CCl₃, —CBr₃, —Cl₃, —OH, —SH, —NH₂, —CN, —NO₂, —COOH, —R^{α}—O—R^{β}, —R^{α}—S—R^{β}, —R^{α}—SO—R^{β}, —R^{α}SO₂—R^{β}, —R^{α}—SO₂—OR^{β}, —R^{α}O—SO₂—R^{β}, —R^{α}—SO₂—N(R^{β})₂, —R^{α}—NR^{β}—SO₂—R^{β}, —R^{α}O—SO₂—OR^{β}, —R^{α}O—SO₂—N(R^{β})₂, —R^{α}—NR^{β}—SO₂—OR^{β}, —R^{α}—NR^{β}—SO₂—N(R^{β})₂, —R^{α}—N(R^{β})₂, —R^{α}—N(R^{β})₃⁺, —R^{α}—P(R^{β})₂, —R^{α}—Si(R^{β})₃, —R^{α}—CO—R^{β}, —R^{α}—CO—OR^{β}, —R^{α}O—CO—R^{β}, —R^{α}—CO—N(R^{β})₂, —R^{α}—NR^{β}—CO—R^{β}, —R^{α}O—CO—OR^{β}, —R^{α}O—CO—N(R_{β})₂, —R^{α}—NR^{β}—CO—OR^{β}, —R^{α}—NR^{β}—CO—N(R^{β})₂, —R^{α}—CS—R^{β}, —R^{α}—CS—OR^{β}, —R^{α}O—CS—R^{β}, —R^{α}—CS—N(R^{β})₂. —R^{α}—NR^{β}—CS—R^{β}, —R^{α}O—CS—OR^{β}, —R^{α}O—CS—N(R^{β})₂, —R^{α}—NR^{β}—CS—OR^{β}, —R^{α}—NR^{β}—CS—N(R^{β})₂, —R^{β}, a bridging substituent such as —O—, —S—, —NR^{β}— or —R^{α}—, or a π-bonded substituent such as ═O, ═S or ═NR^{β}. In this context, —R^{α}— is independently a chemical bond, or a C₁-C₁₀ alkylene, alkenylene or C₁-C₁₀ alkynylene group. —R^{β} is independently hydrogen, unsubstituted C₁-C₆ alkyl or unsubstituted C₆-C₁₀ aryl. Optional substituent(s) are taken into account when calculating the total number of carbon atoms in the parent group substituted with the optional substituent(s). Preferably an optionally substituted group is not substituted with a bridging substituent. Preferably an optionally substituted group is not substituted with a π-bonded substituent. Preferably a substituted group comprises 1, 2 or 3 substituents, more preferably 1 or 2 substituents, and even more preferably 1 substituent.

For the purposes of the present invention, the pregabalin is “racemic”, if it comprises the two enantiomers in a ratio of from 60:40 to 40:60, preferably in a ratio of about 50:50. Similarly, the reaction intermediates used herein, such as intermediates (III), (IV), (V) and (VI), are “racemic”, if they comprise the two enantiomers in a ratio of from 60:40 to 40:60, preferably in a ratio of about 50:50.

The pregabalin is “enantiomerically enriched”, if it comprises 60% or more of only one stereoisomer. Similarly, the reaction intermediates used herein, such as intermediates (IIIa), (IIIb), (IVa), (Va) and (VIa), are “enantiomerically pure”, if they comprise 60% or more of only one stereoisomer.

The pregabalin is “enantiomerically pure”, if it comprises 95% or more of only one stereoisomer, preferably 98% or more, preferably 99% or more, preferably 99.5% or more, preferably 99.9% or more. Similarly, the reaction intermediates used herein, such as intermediates (IIIa), (IIIb), (IVa), (Va) and (VIa), are “enantiomerically pure”, if they comprise 95% or more of only one stereoisomer, preferably 98% or more, preferably 99% or more, preferably 99.5% or more, preferably 99.9% or more.

For the purposes of the present invention, the pregabalin is “substantially free” of lactam impurity, if it comprises less than 3% lactam impurity, preferably less than 2%, preferably less than 1%, preferably less than 0.5%, preferably less than 0.1%.

The “lactam impurity” is the racemic lactam (3) or an enantiomer thereof obtained by an intra-molecular condensation reaction of racemic pregabalin (1) or pregabalin (2).

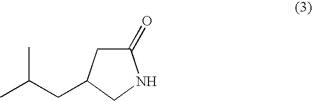

### SUMMARY OF THE INVENTION

The present invention provides an efficient, simple and non-hazardous process for the preparation of pregabalin (2).

The present invention further provides an efficient alternative method for the preparation of racemic pregabalin (1).

A first aspect of the current invention provides a process comprising one or more steps selected from:
(a) the reaction of 4-methyl-2-pentanone (I) with the compound X-G to give the keto intermediate (II):

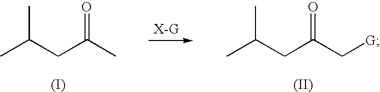

and/or
(b) the reduction of the keto intermediate (II) to the hydroxy intermediate (III):

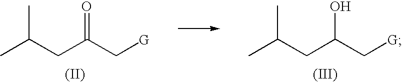

and/or
(c) the displacement of the hydroxyl group of intermediate (III) by a group Y to give intermediate (IV), or the activation of the hydroxyl group of intermediate (III) to give intermediate (V):

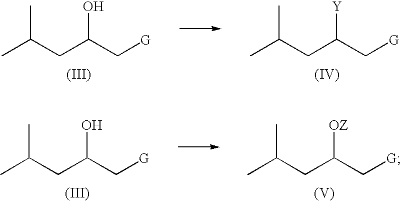

and/or
(d) the reaction of intermediate (IV) or (V) with nitromethane in the presence of a base to give the nitro-derivative (VI):

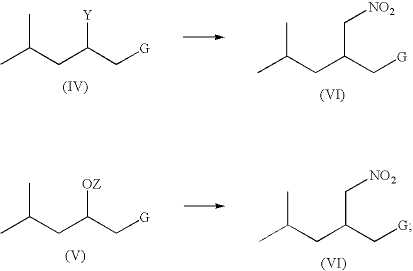

wherein:

X is a suitable leaving group such as a halo, alkoxy, —O-acyl, thio or sulfonate group,

G is a carboxylic acid group or a functional group that is readily converted into a carboxylic acid group,

Y is a suitable leaving group such as a halo group, and

Z is any group that is capable of enhancing the capacity of a hydroxyl group as a leaving group, such as an acyl or sulfonyl group.

The process may comprise one, two, three or four of steps (a)-(d). In a preferred embodiment, the process comprises step (b): the reduction of the keto intermediate (II) to the hydroxy intermediate (III). More preferably, the process comprises an asymmetric reduction of the keto intermediate (II) to the hydroxy intermediate (III).

In one embodiment of the first aspect of the current invention, the process is for the preparation of racemic pregabalin (1), or enantiomerically enriched or enantiomerically pure (S)-(+)-3-aminomethyl-5-methyl-hexanoic acid (2):

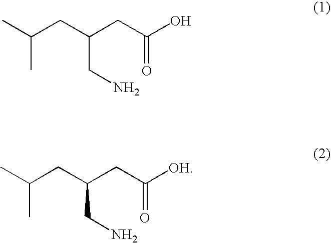

In one embodiment of the first aspect of the current invention, (S)-(+)-3-aminomethyl-5-methyl-hexanoic acid (2) or any of the reaction intermediates are prepared in enantiomerically enriched or enantiomerically pure form.

The group G is preferably a carboxylic ester (e.g. an alkoxycarbonyl) group or another group which can be readily converted to a carboxylic acid group such as a nitrile, a phenyl, an oxazine, an optionally protected aldehyde or ketone, an alkene, an oxazole, an oxazoline, an ortho-ester, a borane or diborane, a nitro, a hydroxy or an alkoxy group. Other examples of such groups are outlined in the reference text book “Protective Groups in Organic Synthesis” by T. W. Greene and P. G. M. Wuts (Wiley-Interscience, 3^{rd} edition, 1999), which is incorporated herein by reference.

The group G is preferably a carboxylic ester group represented by the formula —CO₂R¹, wherein R¹ is selected from an optionally substituted alkyl, alkenyl, alkynyl, aryl, arylalkyl, arylalkenyl, arylalkynyl or silyl group. R¹ is more preferably an alkyl or arylalkyl group and is most preferably a methyl, ethyl or benzyl group.

In one embodiment of the first aspect of the present invention, G is chiral. Where G is a carboxylic ester group represented by the formula —CO₂R¹, R¹ may be chiral, for example, R¹ may be 1-(S)-methyl-n-propyl. The use of a chiral group G allows for the generation of diastereoisomers, rather than enantiomers, in a non-asymmetric reduction of the keto intermediate (II) to the hydroxy intermediate (III).

In another embodiment of the first aspect of the present invention, X is selected from a halo group, or an optionally substituted alkoxy or —O-acyl group. Preferably, where G is a carboxylic ester group represented by the formula —CO₂R¹, X is —OR¹, i.e. the compound X-G is:

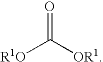

Preferably, Y is selected from —Cl, —Br or —I. Most preferably Y is —Br.

In yet another embodiment of the first aspect of the present invention, Z is selected from a —SO₂R², —SO₂OR², —NO₂, —COR², —P(═O)(OR²)₂ or —B(OR²)₂ group, wherein each R² is independently selected from hydrogen, a halogen, or an optionally substituted alkyl, alkenyl, alkynyl, aryl, arylalkyl, arylalkenyl or arylalkynyl group, and wherein any two R² groups may together with the atoms to which they are attached form a ring. Preferably Z is selected from a —SO₂R² or —SO₂OR² group, preferably wherein R² is independently selected from a halogen, or an alkyl, aryl or arylalkyl group optionally substituted with one or more groups selected from —F, —Cl, —Br or —NO₂. More preferably still, —OZ is selected from a tosylate, brosylate, nosylate, mesylate, tresylate, nonaflate or triflate group. Most preferably —OZ is a triflate group.

In one embodiment of the first aspect of the present invention, 4-methyl-2-pentanone (I) is reacted with the compound X-G in the presence of a base such as sodium hydride, potassium hydride, n-butyl lithium, t-butyl lithium, lithium diisopropylamide or lithium hexamethyldisilylazide. Preferably sodium hydride is used.

A preferred process according to the first aspect of the invention is when the keto compound (II) is reduced to the hydroxy compound (III) with a reducing agent selected from a borohydride, a cyanoborohydride, diborane or another hydride reducing agent. A particularly preferred reducing agent is sodium borohydride.

Another preferred process according to the first aspect of the invention comprises an asymmetric reduction of keto intermediate (II) to hydroxy intermediate (III). The asymmetric reduction may produce the hydroxyl intermediate (IIIa) or the hydroxyl intermediate (Mb) as the major component. Preferably the asymmetric reduction produces the hydroxyl intermediate (Ma) as the major component.

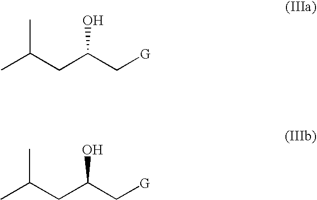

A preferred process is when the asymmetric reduction is achieved using an enzyme. A preferred enzyme is Baker's yeast, particularly a Baker's yeast of the type Mauri.

Another preferred process is when the asymmetric reduction is achieved using catalytic hydrogenation. The catalytic hydrogenation is preferably carried out using a metal catalyst, such as a ruthenium complex. A particularly preferred catalyst is [(S)Ru(BINAP)Cl₂]₂.NEt₃.

One embodiment of the first aspect of the present invention involves the separation of an epimeric mixture of any of the intermediates (III), (IV), (V) or (VI). Preferably the process comprises the separation of hydroxy intermediate (IIIa) from hydroxy intermediate (IIIb). Separation of the epimers at this stage is particularly advantageous since it allows the generation of a single enantiomer of pregabalin from both epimers via complementary routes, as explained below. Thus, separation at this stage avoids the need for one of the epimers to be discarded.

The separation may typically involve the separation of enantiomers. This may be achieved using any technique known to those skilled in the art, such as by the use of chiral chromatography or by classical resolution techniques such as via the generation of diastereomeric salts.

Alternatively, where G is chiral the epimers will be diastereoisomers and consequently the separation may be performed readily by virtue of their differing physical properties. Again, any technique known to those skilled in the art for separating diastereoisomers may be used, such as conventional (i.e. non-chiral) chromatography or re-crystallisation.

In one embodiment of the first aspect of the present invention, intermediate (IV) is generated from intermediate (III) via an S_{N}2 displacement of an activated hydroxyl group by Y⁻. Preferably the activated hydroxyl group is generated in-situ.

Preferably when Y is a halo group, intermediate (IV) is generated from intermediate (III) using Y₂ and R^{x}₃P, or using HY, PY₃, PY₅, an N-halosuccinimide or SOY₂, wherein each Rx is independently selected from an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton. Preferably R^{x}₃P is triphenylphosphine. Alternatively when Y is a halo group, intermediate (IV) may be generated from intermediate (III) using an azidodicarboxylate (such as diethyl azidodicarboxylate), an alkyl halide (such as methyl iodide) and R^{x}₃P (such as triphenylphosphine), wherein R^{x} is as defined above.

In a particularly preferred embodiment of the first aspect of the present invention, intermediate (IVa) is generated from intermediate (IIIa):

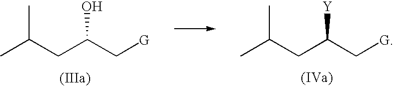

In another, alternative or complementary embodiment of the first aspect of the present invention, intermediate (V) is generated from intermediate (III). Preferably, intermediate (Va) is generated from intermediate (IIIb):

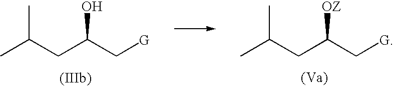

In one embodiment of the first aspect of the present invention, the base used in step (d) is an organic base such as an alkali metal alkoxide (preferably a tertiary alkoxide such as sodium or potassium t-butoxide), or a tertiary amine such as DBU (1,8-diazabicyclo[5.4.0]undec-7-ene), triethylamine, N,N-diisopropyl ethyl amine, DBN (1,5-diazabicyclo[4.3.0]non-5-ene), or DMAP (4-(dimethylamino)pyridine), or an inorganic base such as an alkali metal carbonate (such as sodium or potassium carbonate), or an alkali metal hydroxide (such as sodium or potassium hydroxide). Preferably the base used in step (d) is DBU.

In a particularly preferred embodiment of the first aspect of the present invention, the nitro-derivative (VI) generated in step (d) is nitro-derivative (VIa). The nitro-derivative (VIa) may be generated from intermediate (IVa):

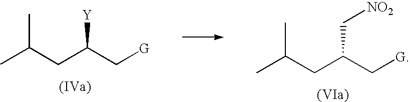

Alternatively, the nitro-derivative (VIa) may be generated from intermediate (Va):

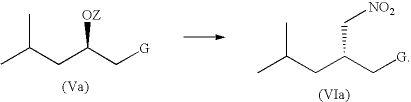

In one embodiment of the first aspect of the present invention, the process further comprises:
(e) the conversion of group G into a carboxylic acid group or a salt thereof; and/or
(f) the reduction of the —NO₂ group to a —NH₂ group or a salt thereof.

Where group G is a carboxylic ester group represented by the formula —CO₂R¹ as defined above, it may be converted into a —CO₂H group by any of a large number of techniques known to those skilled in the art, as exemplified for instance in the reference text book “Protective Groups in Organic Synthesis” by T. W. Greene and P. G. M. Wuts (Wiley-Interscience, 3^{rd} edition, 1999), which is incorporated herein by reference. Representative methods of deprotecting or hydrolysing such an ester are also listed in the detailed description of the invention below. Preferably, however, in accordance with the first aspect of the present invention, the ester is hydrolysed, most preferably using LiOH.

In a preferred embodiment of the first aspect of the present invention, step (f) is performed after step (e).

The reduction of the —NO₂ group to a —NH₂ group may be performed by any number of techniques known to those skilled in the art for the reduction of aliphatic nitro groups to amine groups, several of which are discussed below in the detailed description of the invention. Preferably, however, in accordance with the first aspect of the present invention, the reduction of the —NO₂ group to a —NH₂ group is performed using catalytic hydrogenation, preferably over Pd/C.

Where racemic pregabalin (1) is prepared according to the first aspect of the invention, it can be subsequently resolved to afford (S)-(+)-3-aminomethyl-5-methyl-hexanoic acid (2). Alternatively any of the intermediates obtained can be resolved, for example, the intermediate obtained from step (e) or the intermediate obtained from step (f).

A second aspect of the current invention provides a compound selected from:

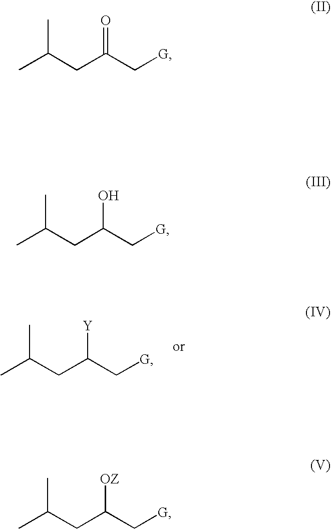

or a salt, tautomer, or stereoisomer thereof, wherein G, Y and Z are as defined in the first aspect of the present invention.

A third aspect of the current invention provides (S)-(+)-3-aminomethyl-5-methyl-hexanoic acid, prepared by a process according to the first aspect of the invention.

A fourth aspect of the current invention provides enantiomerically pure (S)-(+)-3-aminomethyl-5-methyl-hexanoic acid.

A fifth aspect of the current invention provides enantiomerically pure (S)-(+)-3-aminomethyl-5-methyl-hexanoic acid, prepared by a process according to the first aspect of the invention.

A sixth aspect of the current invention provides a pharmaceutical composition comprising the (S)-(+)-3-aminomethyl-5-methyl-hexanoic acid according to the third, fourth or fifth aspect of the invention.

A seventh aspect of the current invention provides the (S)-(+)-3-aminomethyl-5-methyl-hexanoic acid according to the third, fourth or fifth aspect of the invention, for use in medicine, such as for treating or preventing epilepsy, pain, neuropathic pain, cerebral ischaemia, depression, psychoses, fibromyalgia or anxiety.

An eighth aspect of the current invention provides the use of the (S)-(+)-3-aminomethyl-5-methyl-hexanoic acid according to the third, fourth or fifth aspect of the invention, for the manufacture of a medicament for the treatment or prevention of epilepsy, pain, neuropathic pain, cerebral ischaemia, depression, psychoses, fibromyalgia or anxiety.

An ninth aspect of the current invention provides a method of treating or preventing epilepsy, pain, neuropathic pain, cerebral ischaemia, depression, psychoses, fibromyalgia or anxiety, comprising administering to a patient in need thereof a therapeutically or prophylactically effective amount of the (S)-(+)-3-aminomethyl-5-methyl-hexanoic acid according to the third, fourth or fifth aspect of the invention. Preferably the patient is a mammal, preferably a human.

### DETAILED DESCRIPTION OF THE INVENTION

A first aspect of the current invention provides a process for the preparation of racemic pregabalin (1) or (S)-(+)-3-aminomethyl-5-methyl-hexanoic acid (2), comprising the reduction of keto intermediate (II) to the hydroxy intermediate (III) or (IIIa), wherein the group G is a carboxylic acid group or a functional group that is readily converted into a carboxylic acid group.

The keto intermediate (II) is preferably prepared, as outlined in Scheme 1, by reaction of the anion of 4-methyl-2-pentanone with the compound X-G, wherein G is as defined above and X is a suitable leaving group such as a halo group, an alkoxy group or a alkyl or aryl sulfonate group. Preferably, the leaving group X is an alkoxy group.

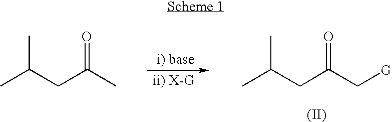

Alternatively, the leaving group X is a halo or sulfonate group. When X is a halo group, it may be a chloro, bromo or iodo group, preferably a bromo group. When X is a sulfonate group, it may be a mesylate, triflate, tosylate or besylate group.

The anion of 4-methyl-2-pentanone can be generated with any suitable base, but is preferably prepared using sodium hydride.

A particularly preferred embodiment of the invention is when the group G is an ethoxycarbonyl (ethyl ester) group and the group X is an ethoxy group, such that the compound X-G is the commercially available reagent diethyl carbonate.

A preferred embodiment of the first aspect of the invention for the preparation of racemic pregabalin (1) is illustrated in Scheme 2. Thus, 4-methyl-2-pentanone is reacted with sodium hydride and diethyl carbonate and the resulting ethyl 5-methyl-3-oxo-hexanoate is reduced with sodium borohydride to afford racemic ethyl 5-methyl-3-hydroxy-hexanoate. This hydroxy intermediate is then converted to the bromo hexanoate, which is subsequently reacted with nitromethane, to afford racemic ethyl 5-methyl-3-nitromethyl-hexanoate. Subsequent saponification of the ester to the carboxylic acid and reduction of the nitro group by hydrogenation with a palladium on carbon catalyst affords racemic pregabalin (1). The above process is very efficient and affords racemic pregabalin (1) in high yield and in high purity. A further advantage of this process is that it does not use hazardous reagents such as potassium cyanide.

Preferably, the racemic pregabalin (1) is obtained in a yield of 60% or more, preferably 65% or more, preferably 70% or more. Preferably, the racemic pregabalin (1) is obtained substantially free of lactam impurity (3).

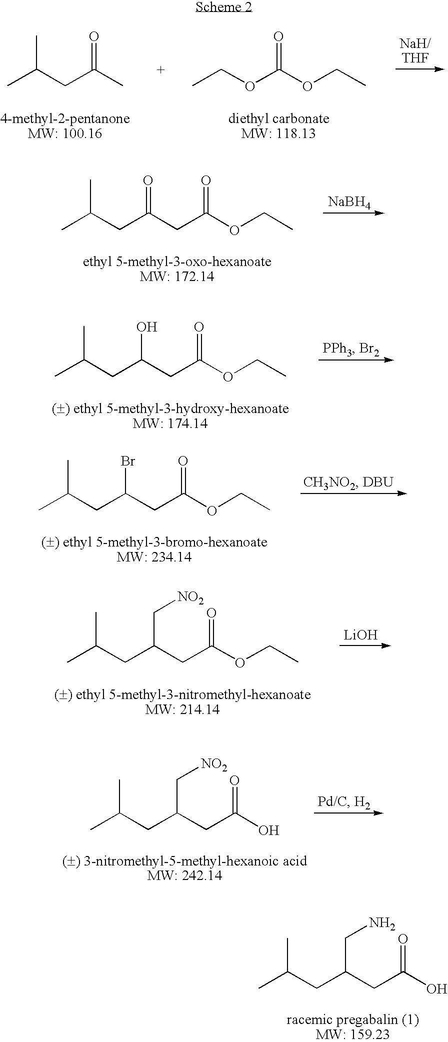

If required, the conversion of racemic pregabalin (1) to pregabalin (2) can be done by following well-established and reported routes of resolution. For example, U.S. Pat. No. 5,637,767, which is herein incorporated by reference in its entirety, reports the resolution of racemic pregabalin (1) to pregabalin (2) by selective crystallisation with (S)- or (R)-mandelic acid.

Alternatively, pregabalin (2) may be prepared via the resolution of one of the earlier intermediates such as by the resolution of racemic ethyl 5-methyl-3-hydroxy-hexanoate. The (S) ethyl 5-methyl-3-hydroxy-hexanoate may be converted into pregabalin (2) as described in relation to Scheme 4 below. In a complementary route, the (R) ethyl 5-methyl-3-hydroxy-hexanoate may be converted into pregabalin (2) by activating the hydroxyl group, e.g. by converting it into a triflate group, and then reacting the resultant triflate with nitromethane to give the desired (S) ethyl 5-methyl-3-nitromethyl-hexanoate with inversion of configuration at the stereocentre. This is illustrated in Scheme 3 below.

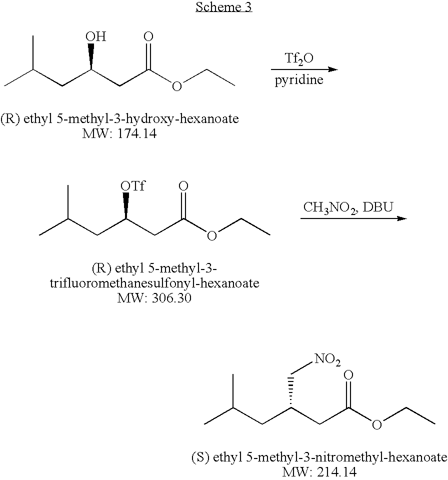

The (S) ethyl 5-methyl-3-nitromethyl-hexanoate may then be converted into pregabalin (2) as described in relation to Scheme 4 below.

However, alternatively still, the process according to the present invention can be varied to prepare pregabalin (2) directly, without the need for resolution, via an asymmetric reduction of a keto intermediate, such as ethyl 5-methyl-3-oxo-hexanoate.

A particularly preferred embodiment of the first aspect of the invention is outlined in Scheme 4. Thus, 4-methyl-2-pentanone is reacted with sodium hydride and diethyl carbonate and the resulting ethyl 5-methyl-3-oxo-hexanoate is reduced with either Baker's yeast or by catalytic hydrogenation with the catalyst [(S)Ru(BINAP)Cl₂]₂.NEt₃ to afford (S) ethyl 5-methyl-3-hydroxy-hexanoate. This enantiomerically pure hydroxy intermediate is then converted to the bromo hexanoate, which is subsequently reacted with nitromethane, to afford (S) ethyl 5-methyl-3-nitromethyl-hexanoate. Subsequent saponification of the ester to the carboxylic acid and reduction of the nitro group by hydrogenation with a palladium on carbon catalyst affords pregabalin (2). The above process is very efficient and affords enantiomerically pure pregabalin (2) in high yield and in high chemical and optical purity.

Preferably, the pregabalin (2) is obtained in a yield of 60% or more, preferably 65% or more, preferably 70% or more. Preferably, the pregabalin (2) is obtained substantially free of lactam impurity (3) and is enantiomerically pure.

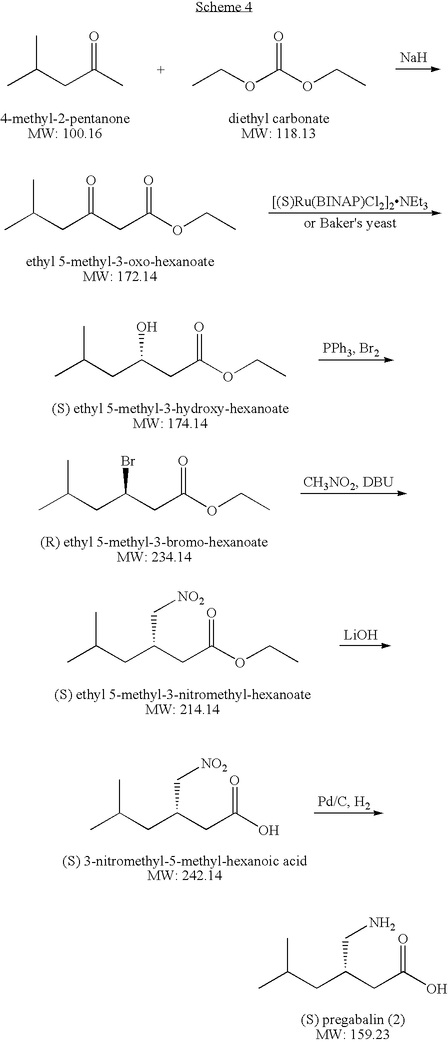

The reagents and solvents illustrated in Schemes 2 to 4 are merely illustrative of the current invention and the reaction schemes are not limited by these reagents or solvents. Any suitable alternatives can be used as outlined below.

Generation of the anion of 4-methyl-2-pentanone is preferably achieved with sodium hydride but other suitable bases can be used, such as potassium hydride, n-butyl lithium, t-butyl lithium, lithium diisopropylamide or lithium hexamethyldisilylazide.

Conversion of the hydroxy intermediate to the bromo intermediate is preferably performed using triphenylphosphine/bromine, but other suitable reagents, such as HBr, PBr₃, PBr₅, N-bromosuccinimide or SOBr₂, may also be used.

Aliphatic nitro groups like those in 3-nitromethyl-5-methyl-hexanoic acid can be reduced to amine groups by many reducing agents including catalytic hydrogenation (using hydrogen gas and a catalyst such as Pt, Pt/C, PtO₂, Pd, Pd/C, Rh, Ru, Ni or Raney Ni); Zn, Sn or Fe and an acid; AlH₃-AlCl₃; hydrazine and a catalyst; [Fe₃(CO)₁₂]-methanol; TiCl₃; hot liquid paraffin; formic acid or ammonium formate and a catalyst such as Pd/C; LiAlH₄; and sulfides such as NaHS, (NH₄)₂S or polysulfides.

Esters like those in 3-nitromethyl-5-methyl-hexanoic acid ester can be deprotected or hydrolysed to give the free carboxylic acids under a number of conditions. Many of these preferred esters can be deprotected under acidic conditions (using, for example, CH₃CO₂H, CF₃CO₂H, HCO₂H, HCl, HBr, HF, CH₃SO₃H and/or CF₃SO₃H); or under basic conditions (using, for example, LiOH, NaOH, KOH, Ba(OH)₂, K₂CO₃ or Na₂S). Esters, such as benzyl, carbobenzoxy (Cbz), trityl (triphenylmethyl), benzyloxymethyl, phenacyl, diphenylmethyl and 4-picolyl esters, can be deprotected by catalytic hydrogenolysis (using hydrogen gas and a catalyst such as Pt, Pt/C, PtO₂, Pd, Pd/C, Rh, Ru, Ni or Raney Ni), by catalytic transfer hydrogenolysis (using a hydrogen donor such as cyclohexene, 1,4-cyclohexadiene, formic acid, ammonium formate or cis-decalin and a catalyst such as Pd/C or Pd); by electrolytic reduction; by irradiation; using a Lewis acid (such as AlCl₃, BF₃, BF₃-Et₂O, BBr₃ or Me₂BBr); or using sodium in liquid ammonia. Benzyl esters can also be deprotected using aqueous CuSO₄ followed by EDTA; NaHTe in DMF; or Raney Ni and Et₃N. Carbobenzoxy esters can also be deprotected using Me₃SiI; or LiAlH₄ or NaBH₄ and Me₃SiCl. Trityl esters can also be deprotected using MeOH or H₂O and dioxane. Phenacyl esters can also be deprotected using Zn and an acid such as AcOH; PhSNa in DMF; or PhSeH in DMF.

A sixth aspect of the current invention provides a pharmaceutical composition comprising the (S)-(+)-3-aminomethyl-5-methyl-hexanoic acid according to the third, fourth or fifth aspect of the invention.

The pharmaceutical composition according to the sixth aspect of the current invention can be a solution or suspension form, but is preferably a solid oral dosage form. Preferred dosage forms in accordance with the invention include tablets, capsules and the like which, optionally, may be coated if desired. Tablets can be prepared by conventional techniques, including direct compression, wet granulation and dry granulation. Capsules are generally formed from a gelatine material and can include a conventionally prepared granulate of excipients in accordance with the invention.

The pharmaceutical composition according to the current invention typically comprises one or more conventional pharmaceutically acceptable excipient(s) selected from the group comprising a filler, a binder, a disintegrant and a lubricant, and optionally further comprises at least one excipient selected from colouring agents, adsorbents, surfactants, film formers and plasticizers.

As described above, the stable pharmaceutical composition of the invention typically comprises one or more fillers such as microcrystalline cellulose, lactose, sugars, starches, modified starches, mannitol, sorbitol and other polyols, dextrin, dextran or maltodextrin; one or more binders such as lactose, starches, modified starch, maize starch, dextrin, dextran, maltodextrin, microcrystalline cellulose, sugars, polyethylene glycols, hydroxypropyl cellulose, hydroxypropyl methyl cellulose, ethyl cellulose, hydroxyethyl cellulose, methyl cellulose, carboxymethyl cellulose, gelatine, acacia gum, tragacanth, polyvinylpyrrolidone or crospovidone; one or more disintegrating agents such as croscarmellose sodium, cross-linked polyvinylpyrrolidone, crospovidone, cross-linked carboxymethyl starch, starches, microcrystalline cellulose or polyacrylin potassium; one or more different glidants or lubricants such as magnesium stearate, calcium stearate, zinc stearate, calcium behenate, sodium stearyl fumarate, talc, magnesium trisilicate, stearic acid, palmitic acid, carnauba wax or silicon dioxide.

If required, the pharmaceutical composition of the invention may also include surfactants and other conventional excipients. Typical surfactants that may be used are ionic surfactants such as sodium lauryl sulfate or non-ionic surfactants such as different poloxamers (polyoxyethylene and polyoxypropylene copolymers), natural or synthesized lecithins, esters of sorbitan and fatty acids (such as Spano®), esters of polyoxyethylene sorbitan and fatty acids (such as Tween®), polyoxyethylated hydrogenated castor oil (such as Cremophor®), polyoxyethylene stearates (such as Brij®), dimethylpolysiloxane or any combination of the above mentioned surfactants.

If the solid pharmaceutical formulation is in the form of coated tablets, the coating may be prepared from at least one film-former such as hydroxypropyl methyl cellulose, hydroxypropyl cellulose or methacrylate polymers which optionally may contain at least one plasticizer such as polyethylene glycols, dibutyl sebacate, triethyl citrate, and other pharmaceutical auxiliary substances conventional for film coatings such as pigments, fillers and others.

The following paragraphs enumerated consecutively from 1 through 63 provide for various aspects of the present invention. In one embodiment, the present invention provides:

1. A process comprising one or more steps selected from:
(a) the reaction of 4-methyl-2-pentanone (I) with the compound X-G to give the keto intermediate (II):

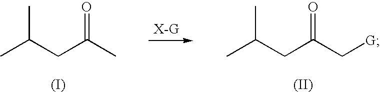

and/or
(b) the reduction of the keto intermediate (II) to the hydroxy intermediate (III):

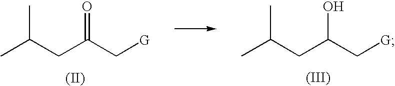

and/or
(c) the displacement of the hydroxyl group of intermediate (III) by a group Y to give intermediate (IV), or the activation of the hydroxyl group of intermediate (III) to give intermediate (V):

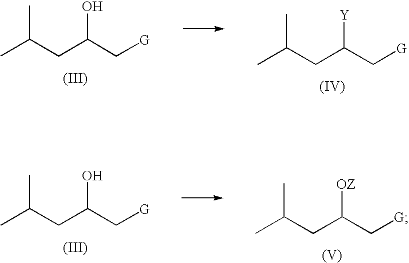

and/or
(d) the reaction of intermediate (IV) or (V) with nitromethane in the presence of a base to give the nitro-derivative (VI):

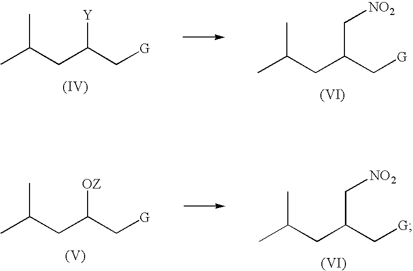

wherein:

X is a suitable leaving group such as a halo, alkoxy, —O-acyl, thio or sulfonate group,

G is a carboxylic acid group or a functional group that is readily converted into a carboxylic acid group,

Y is a suitable leaving group such as a halo group, and

Z is any group that is capable of enhancing the capacity of a hydroxyl group as a leaving group, such as an acyl or sulfonyl group.

2. A process according to paragraph 1, comprising the reduction of the keto intermediate (II) to the hydroxy intermediate (III).
3. A process according to paragraph 2, comprising an asymmetric reduction of the keto intermediate (II) to the hydroxy intermediate (III).
4. A process according to any one of paragraphs 1 to 3, for the preparation of racemic pregabalin (1) or (S)-(+)-3-aminomethyl-5-methyl-hexanoic acid (2):

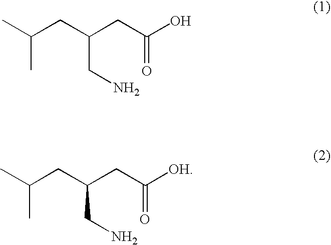

5. A process according to any one of paragraphs 1 to 4, wherein G is chiral.
6. A process according to any one of paragraphs 1 to 5, wherein the group G is a carboxylic ester, a nitrile, a phenyl, an oxazine, an optionally protected aldehyde or ketone, an alkene, an oxazole, an oxazoline, an ortho-ester, a borane or diborane, a nitro, a hydroxy or an alkoxy group.
7. A process according to paragraph 6, wherein the group G is a carboxylic ester group represented by the formula —CO₂R¹, wherein R¹ is selected from an optionally substituted alkyl, alkenyl, alkynyl, aryl, arylalkyl, arylalkenyl, arylalkynyl or silyl group.
8. A process according to paragraph 7, wherein R¹ is an optionally substituted alkyl or arylalkyl group.
9. A process according to paragraph 8, wherein R¹ is a methyl, ethyl or benzyl group.
10. A process according to paragraph 9, wherein R¹ is an ethyl group.
11. A process according to paragraph 7 or 8, wherein R¹ is chiral.
12. A process according to any one of paragraphs 1 to 11, wherein X is selected from a halo group, or an optionally substituted alkoxy or —O-acyl group.
13. A process according to any one of paragraphs 7 to 11, wherein X is —OR¹.
14. A process according to any one of paragraphs 1 to 13, wherein Y is selected from —Cl, —Br or —I.
15. A process according to any one of paragraphs 1 to 14, wherein Z is selected from a —SO₂R², —SO₂OR², —NO₂, —COR², —P(═O)(OR²)₂ or —B(OR²)₂ group, wherein each R² is independently selected from hydrogen, a halogen, or an optionally substituted alkyl, alkenyl, alkynyl, aryl, arylalkyl, arylalkenyl or arylalkynyl group, and wherein any two R² groups may together with the atoms to which they are attached form a ring.
16. A process according to paragraph 15, wherein Z is selected from a —SO₂R² or —SO₂OR² group.
17. A process according to paragraph 16, wherein R² is independently selected from a halogen, or an alkyl, aryl or arylalkyl group optionally substituted with one or more groups selected from —F, —Cl, —Br or —NO₂.
18. A process according to paragraph 17, wherein —OZ is selected from a tosylate, brosylate, nosylate, mesylate, tresylate, nonaflate or triflate group.
19. A process according to any one of paragraphs 1 to 18, wherein 4-methyl-2-pentanone (I) is reacted with the compound X-G in the presence of a base.
20. A process according to paragraph 19, wherein the base is sodium hydride.
21. A process according to any one of paragraphs 1 to 20, wherein the keto compound (II) is reduced to the hydroxy compound (III) with a reducing agent selected from a borohydride, a cyanoborohydride, diborane or another hydride reducing agent.
22. A process according to paragraph 21, wherein the reducing agent is sodium borohydride.
23. A process according to any one of paragraphs 1 to 22, involving an asymmetric reduction of keto intermediate (II) to hydroxy intermediate (III).
24. A process according to paragraph 23, wherein the asymmetric reduction is to hydroxy intermediate (IIIa):

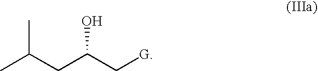

25. A process according to paragraph 23, wherein the asymmetric reduction is to hydroxy intermediate (IIIb):

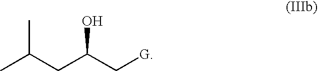

26. A process according to any one of paragraphs 23 to 25, wherein the asymmetric reduction is achieved using an enzyme.
27. A process according to paragraph 26, wherein the enzyme is Baker's yeast.
28. A process according to paragraph 27, wherein the Baker's yeast is of the type Mauri.
29. A process according to any one of paragraphs 23 to 25, wherein the asymmetric reduction is achieved using catalytic hydrogenation.
30. A process according to paragraph 29, wherein the catalyst is a ruthenium complex.
31. A process according to paragraph 30, wherein the catalyst is [(S)Ru(BINAP)Cl₂]₂.NEt₃.
32. A process according to any one of paragraphs 1 to 31, further comprising the separation of hydroxy intermediate (IIIa) from hydroxy intermediate (IIIb).
33. A process according to paragraph 32, wherein the separation is the separation of enantiomers.
34. A process according to paragraph 32, wherein G is chiral and the separation is the separation of diastereoisomers.
35. A process according to any one of paragraphs 1 to 34, wherein intermediate (IV) is generated from intermediate (III) via an S_{N}2 displacement of an activated hydroxyl group by Y⁻.
36. A process according to paragraph 35, wherein the hydroxyl group is activated in-situ.
37. A process according to any one of paragraphs 1 to 36, wherein Y is a halogen and intermediate (IV) is generated from intermediate (III) using Y₂ and R^{x}₃P, or using HY, PY₃, PY₅, an N-halosuccinimide or SOY₂, wherein each R^{x} is independently selected from an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton.
38. A process according to any one of paragraphs 1 to 36, wherein Y is a halogen and intermediate (IV) is generated from intermediate (III) using an azidodicarboxylate, an alkyl halide and R^{x}₃P, wherein each R^{x} is independently selected from an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton.
39. A process according to any one of paragraphs 1 to 38, wherein intermediate (IVa) is generated from intermediate (IIIa):

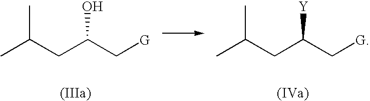

40. A process according to any one of paragraphs 1 to 38, wherein intermediate (V) is generated from intermediate (III).
41. A process according to paragraph 40, wherein intermediate (Va) is generated from intermediate (IIIb):

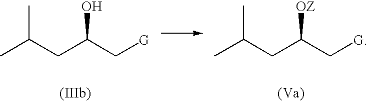

42. A process according to any one of paragraphs 1 to 41, wherein the base used in step (d) is an organic base such as an alkali metal alkoxide, or a tertiary amine such as DBU, triethylamine, N,N-diisopropyl ethyl amine, DBN, or DMAP, or an inorganic base such as an alkali metal carbonate or an alkali metal hydroxide.
43. A process according to paragraph 42, wherein the base used in step (d) is DBU.
44. A process according to any one of paragraphs 1 to 43, wherein the nitro-derivative (VIa) is generated from intermediate (IVa):

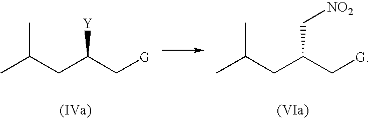

45. A process according to any one of paragraphs 1 to 43, wherein the nitro-derivative (VIa) is generated from intermediate (Va):

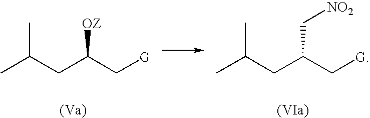

46. A process according to any one of paragraphs 1 to 45, further comprising:
(e) the conversion of group G into a carboxylic acid group or a salt thereof; and/or
(f) the reduction of the —NO₂ group to a —NH₂ group or a salt thereof.
   47. A process according to paragraph 46, wherein the group G is a carboxylic ester group represented by the formula —CO₂R¹, wherein R¹ is selected from an optionally substituted alkyl, alkenyl, alkynyl, aryl, arylalkyl, arylalkenyl, arylalkynyl or silyl group, and wherein the carboxylic acid group or a salt thereof is generated by hydrolysis.
   48. A process according to paragraph 47, wherein LiOH is used to hydrolyse the ester.
   49. A process according to any one of paragraphs 46 to 48, wherein step (f) is performed after step (e).
   50. A process according to any one of paragraphs 46 to 49, wherein the reduction of the —NO₂ group to a —NH₂ group is performed using catalytic hydrogenation.
   51. A process according to paragraph 50, wherein the catalyst is Pd/C.
   52. A process for the preparation of (S)-(+)-3-aminomethyl-5-methyl-hexanoic acid (2), comprising resolution of racemic pregabalin (1) prepared by a process according to any one of the preceding paragraphs.
   53. A compound selected from:

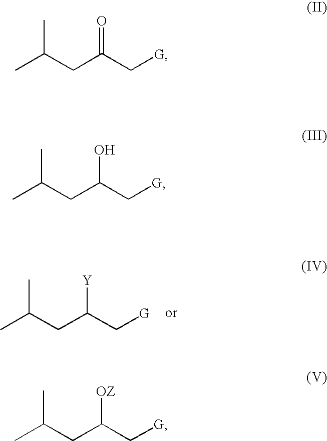

or a salt, tautomer, or stereoisomer thereof, wherein G, Y and Z are as defined in any one of the preceding paragraphs.
54. (S)-(+)-3-aminomethyl-5-methyl-hexanoic acid prepared by a process according to any one of paragraphs 1 to 52.
55. Enantiomerically pure (S)-(+)-3-aminomethyl-5-methyl-hexanoic acid.
56. Enantiomerically pure (S)-(+)-3-aminomethyl-5-methyl-hexanoic acid, prepared by a process according to any one of paragraphs 1 to 52.
57. A pharmaceutical composition comprising the (S)-(+)-3-aminomethyl-5-methyl-hexanoic acid according to any one of paragraphs 54 to 56.
58. (S)-(+)-3-aminomethyl-5-methyl-hexanoic acid according to any one of paragraphs 54 to 56, for use in medicine.
59. (S)-(+)-3-aminomethyl-5-methyl-hexanoic acid according to paragraph 58, for treating or preventing epilepsy, pain, neuropathic pain, cerebral ischaemia, depression, psychoses, fibromyalgia or anxiety.
60. Use of (S)-(+)-3-aminomethyl-5-methyl-hexanoic acid according to any one of paragraphs 54 to 56, for the manufacture of a medicament for the treatment or prevention of epilepsy, pain, neuropathic pain, cerebral ischaemia, depression, psychoses, fibromyalgia or anxiety.
61. A method of treating or preventing epilepsy, pain, neuropathic pain, cerebral ischaemia, depression, psychoses, fibromyalgia or anxiety, comprising administering to a patient in need thereof a therapeutically or prophylactically effective amount of (S)-(+)-3-aminomethyl-5-methyl-hexanoic acid according to any one of paragraphs 54 to 56.
62. A method according to paragraph 61, wherein the patient is mammal.
63. A method according to paragraph 62, wherein the mammal is a human.

### EXAMPLES

Schemes 2, 3 and 4 illustrate non-limiting examples of the current invention and experimental details for these examples are given below.

### Ethyl 5-methyl-3-oxo-hexanoate

NaH (2eq) was taken in THF (5vol) at 20-25° C. and diethyl carbonate (1.35eq) was added. A solution of 4-methyl-2-pentanone (1eq) in diethyl carbonate (2.98vol) was gradually added and the mixture was heated at reflux. After 4 hours, the reaction mixture was added to ice cold water (10vol), neutralized with glacial acetic acid (1.6vol) at 0-10° C. and stirred for 20 minutes. The mixture was extracted with ethyl acetate and the combined ethyl acetate extracts were washed with 10% sodium bicarbonate solution (10vol) and water. The ethyl acetate layer was removed under vacuum at 50° C. The product was obtained as brown coloured oil. Molar yield=95%.

### (±) Ethyl 5-methyl-3-hydroxy-hexanoate

Sodium borohydride (0.8eq) was added to ethanol (5vol) at 0° C. slowly and then ethyl 5-methyl-3-oxo-hexanoate (1eq) was added. The mixture was warmed to 25-30° C. and stirred for 3 hours. After completion of the reaction, the ethanol was removed under vacuum at 50° C., and aqueous HCl (1:1 mixture) was added to adjust the pH to about 3. The aqueous mixture was extracted with ethyl acetate and the organic extracts were washed with water. The ethyl acetate was removed to obtain the product as colourless oil. Molar yield=84%.

### (±) Ethyl 5-methyl-3-bromo-hexanoate

Triphenylphosphine (1.1eq) was added to DCM (5vol) and cooled to 0° C. Bromine (1.1eq) was added to the above solution at 0° C. and stirred at that temperature for 10-15 minutes. (±) Ethyl 5-methyl-3-hydroxy-hexanoate (1eq) was added to the above white slurry and stirred for 30 minutes. After completion of the reaction, water was added and the DCM layer was separated. The aqueous layer was re-extracted with DCM. Concentration of the combined DCM layers under vacuum gave the crude product. Column chromatography of the crude product using hexane/ethyl acetate yielded the product as a yellow liquid. Molar yield=70%.

### (±) Ethyl 5-methyl-3-nitromethyl-hexanoate

To a solution of (±) ethyl 5-methyl-3-bromo-hexanoate (1eg) in nitromethane (4vol) at 0-5° C. was added DBU (1.05eq) dropwise over 30 minutes. After completion of the addition, the reaction mixture was allowed to attain 25-30° C. and stirred at this temperature for 2 hours. After completion of the reaction, the reaction mixture was poured into a mixture of conc. HCl(0.4vol) and water (15vol) and stirred for 15 minutes. The reaction mixture was extracted with ethyl acetate and the combined organic extracts were washed with water. The combined organic layer was dried over sodium sulfate and concentrated under reduced pressure to give the product as yellow oil. Molar yield=96%.

### (±) 3-Nitromethyl-5-methyl-hexanoic acid

(±) Ethyl 5-methyl-3-nitromethyl-hexanoate (1eq) was dissolved in THF-water (10vol, 2:1), lithium hydroxide (2.5eq) was added and the reaction mixture stirred for 3-4 hours. The reaction mass was concentrated to remove THF at 35° C. under reduced pressure. Water (5vol) was added to the aqueous mass and extracted with ethyl acetate, acidified with conc. HCl (1vol) and extracted with DCM. The DCM layer was washed with water (10vol) and concentrated under reduced pressure at 35-40° C. to afford the product as oil. Molar yield=85%.

### (±) Pregabalin (1)

Hydrogen was bubbled through a solution of (±) 3-nitromethyl-5-methyl-hexanoic acid (1eq) in methanol (15vol) in the presence of 60% (w/w) of wet 5% palladium on carbon. After completion of the reaction (5-8 hours), the reaction mixture was filtered through a Celite® bed and the filtrate concentrated under reduced pressure to give (±) pregabalin as an oil/sticky solid. The crude product was crystallized from hot 2-propanol/water 1:1 (10vol) to obtain the product as white solid. Molar yield=37%.

### (S) Ethyl 5-methyl-3-hydroxy-hexanoate

### Enzymatic Reduction

Mauri yeast dry powder (200 times w/w) was added to a water (800vol) and allyl alcohol (5.9vol) mixture at 25-30° C. This was stirred for 24 hours before addition of ethyl 5-methyl-3-oxo-hexanoate. Stirring was continued for another 24 hours before filtering the reaction mixture through a Celite® bed, extracting the filtrate with ethyl acetate (4×80vol) and removing the solvent under vacuum to afford a colourless oil. Molar yield=50%; Enantiomeric excess>99%.

### Chemical Reduction

[(S)Ru(BINAP)Cl₂]₂.NEt₃(0.00046eq) was taken in methanol (8vol) and conc. HCl (0.005vol) was added under nitrogen. Ethyl 5-methyl-3-oxo-hexanoate was added to the above slurry and hydrogenation was performed at 40° C. and 50 psi. After completion of the reaction, the reaction mass was filtered and concentrated to afford the product as colourless oil. Molar yield=66%; Enantiomeric excess>99%.

### (R) Ethyl 5-methyl-3-bromo-hexanoate

Triphenylphosphine (1.1eq) was added to DCM (5vol) and cooled to 0° C. Bromine (1.1eq) was added to the above solution at 0° C. and stirred at that temperature for 10-15 minutes. (S) Ethyl 5-methyl-3-hydroxy-hexanoate (1eq) was added to the above white slurry and stirred for 30 minutes. After completion of the reaction, water was added and the DCM layer was separated. The aqueous layer was re-extracted with DCM and removal of the combined DCM layer under vacuum gave crude product. Column chromatography of the crude product using hexane/ethyl acetate yielded the product as yellow liquid. Molar yield=73%; Enantiomeric excess>99%.

### (S) Ethyl 5-methyl-3-nitromethyl-hexanoate

To a solution of (R) ethyl 5-methyl-3-bromo-hexanoate (1eq) in nitromethane (4vol) at 0-5° C. was added DBU (1.05eq) dropwise over 30 minutes. After completion of the addition, the reaction mixture was allowed to attain 25-30° C. and stirred at this temperature for 2 hours. After completion of the reaction, the reaction mixture was poured into a mixture of conc. HCl(0.4vol) and water (15vol) and stirred for 15 minutes. The reaction mixture was extracted with ethyl acetate and the combined organic extracts were washed with water. The organic layer was dried over sodium sulfate and concentrated under reduced pressure to give the product as yellow oil. Molar yield=96%; Enantiomeric excess=99%.

### (S) 3-Nitromethyl-5-methyl-hexanoic acid

(S) Ethyl 5-methyl-3-nitromethyl-hexanoate (1eq) was dissolved in THF-water (10vol, 2:1), lithium hydroxide (2.5eq) was added and the reaction mixture stirred for 3-4 hours. The reaction was monitored by TLC. At the end of the reaction, the reaction mass was concentrated to remove THF at 35° C. under reduced pressure. Water (5vol) was added to the aqueous mass and extracted with ethyl acetate, acidified with conc. HCl (1vol) and extracted with DCM. The combined DCM layer was washed with water (10vol). Concentration under reduced pressure at 35-40° C. afforded the product as an oil. Molar yield=85%; Enantiomeric excess>99%.

### Pregabalin (2)

Hydrogen was bubbled through a solution of (S) 3-nitromethyl-5-methyl-hexanoic acid (1eq) in methanol (15vol) in the presence of 60% (w/w) of wet 5% palladium on carbon. After completion of the reaction (5-8 hours), the reaction mixture was filtered through a Celite® bed and the filtrate concentrated under reduced pressure to give pregabalin as an oil/sticky solid. The crude product was crystallized from hot 2-propanol/water 1:1 (10vol) to obtain the product as white solid. Molar yield=35%; Enantiomeric excess>99%; HPLC purity=99.6%.

¹H NMR spectrum (D₂O+1 drop of DCl) ppm: 2.87 (d, J=6.3 Hz, 2H); 2.34 (m, 2H); 2.08 (m, 1H); 1.48 (m, 1H); 1.08 (t, J=7.2 Hz, 2H); 0.73 (d, J=6.6 Hz, 3H); 0.71 (d, J=6.6 Hz, 3H).

Mass Spec (electro spray ionization): (M+H)⁺ 160.2; (M−H₂O+H)⁺ 142.2.

Theoretical Preparation of (R) ethyl 5-methyl-3-trifluoromethanesulfonyl-hexanoate

Pyridine (5eq) is added to a solution of (R) ethyl 5-methyl-3-hydroxy-hexanoate (1eq) in DCM (10vol) under N₂ at −78° C. Tf₂O (2eq) is then added dropwise and the mixture is stirred at −78° C. for a further 20 minutes, before warming to 0° C. and stirring for a further 2-3 hours. The reaction is monitored by TLC. After completion of the reaction, the mixture is diluted with DCM, washed with 0.1M HCl then with water. The organic fraction is dried over MgSO₄, filtered, and the solvent removed under vacuum to give the crude product. Column chromatography of the crude product using hexane/ethyl acetate yields the product.

Theoretical Preparation of (S) ethyl 5-methyl-3-nitromethyl-hexanoate

DBU (1.05eq) is added dropwise over 30 minutes to a solution of (R) ethyl 5-methyl-3-trifluoromethanesulfonyl-hexanoate (1eq) in nitromethane (4vol) at 0-5° C. After completion of the addition, the reaction mixture is allowed to attain 25-30° C. and the mixture is stirred at this temperature for 2 hours. After completion of the reaction, the reaction mixture is poured into a mixture of conc. HCl (0.4vol) and water (15vol) and stirred for 15 minutes. The reaction mixture is extracted with ethyl acetate and the combined organic extracts are washed with water. The organic layer is dried over sodium sulphate and concentrated under reduced pressure to give the product.

### (±) Ethyl 5-methyl-3-nitromethyl-hexanoate

To a solution of (±) ethyl 5-methyl-3-bromo-hexanoate (1eq) in nitromethane (4vol) at 0-5° C. was added DBU (1.05eq) dropwise over 30 minutes. After completion of the addition, the reaction mixture was allowed to attain 25-30° C. and stirred at this temperature for 2 hours. After completion of the reaction, the reaction mixture was poured into a mixture of conc. HCl (0.4vol) and water (15vol) and stirred for 15 minutes. The reaction mixture was extracted with ethyl acetate and the combined organic extracts were washed with water. The combined organic layer was dried over sodium sulfate and concentrated under reduced pressure to give the product as yellow oil. Molar yield=96%.

### (±) 3-Nitromethyl-5-methyl-hexanoic acid

(±) Ethyl 5-methyl-3-nitromethyl-hexanoate (1eq) was dissolved in THF-water (10vol, 2:1), lithium hydroxide (2.5eq) was added and the reaction mixture stirred for 3-4 hours. The reaction mass was concentrated to remove THF at 35° C. under reduced pressure. Water (5vol) was added to the aqueous mass and extracted with ethyl acetate, acidified with conc. HCl (1vol) and extracted with DCM. The DCM layer was washed with water (10vol) and concentrated under reduced pressure at 35-40° C. to afford the product as oil. Molar yield=85%.

### (±) Pregabalin (1)

Hydrogen was bubbled through a solution of (±) 3-nitromethyl-5-methyl-hexanoic acid (1eq) in methanol (15vol) in the presence of 60% (w/w) of wet 5% palladium on carbon. After completion of the reaction (5-8 hours), the reaction mixture was filtered through a Celite® bed and the filtrate concentrated under reduced pressure to give (±) pregabalin as an oil/sticky solid. The crude product was crystallized from hot 2-propanol/water 1:1 (10vol) to obtain the product as white solid. Molar yield=37%.

### (S) Ethyl 5-methyl-3-hydroxy-hexanoate

### Enzymatic Reduction

Mauri yeast dry powder (200 times w/w) was added to a water (800vol) and allyl alcohol (5.9vol) mixture at 25-30° C. This was stirred for 24 hours before addition of ethyl 5-methyl-3-oxo-hexanoate. Stirring was continued for another 24 hours before filtering the reaction mixture through a Celite® bed, extracting the filtrate with ethyl acetate (4×80vo1) and removing the solvent under vacuum to afford a colourless oil. Molar yield=50%; Enantiomeric excess>99%.

### Chemical Reduction

[(S)Ru(BINAP)Cl₂]₂.NEt₃ (0.00046eq) was taken in methanol (8vol) and conc. HCl(0.005vol) was added under nitrogen. Ethyl 5-methyl-3-oxo-hexanoate was added to the above slurry and hydrogenation was performed at 40° C. and 50 psi. After completion of the reaction, the reaction mass was filtered and concentrated to afford the product as colourless oil. Molar yield=66%; Enantiomeric excess>99%.

### (R) Ethyl 5-methyl-3-bromo-hexanoate

Triphenylphosphine (1.1eq) was added to DCM (5vol) and cooled to 0° C. Bromine (1.1eq) was added to the above solution at 0° C. and stirred at that temperature for 10-15 minutes. (S) Ethyl 5-methyl-3-hydroxy-hexanoate (1eq) was added to the above white slurry and stirred for 30 minutes. After completion of the reaction, water was added and the DCM layer was separated. The aqueous layer was re-extracted with DCM and removal of the combined DCM layer under vacuum gave crude product. Column chromatography of the crude product using hexane/ethyl acetate yielded the product as yellow liquid. Molar yield=73%; Enantiomeric excess>99%.

### (S) Ethyl 5-methyl-3-nitromethyl-hexanoate

To a solution of (R) ethyl 5-methyl-3-bromo-hexanoate (1eq) in nitromethane (4vol) at 0-5° C. was added DBU (1.05eq) dropwise over 30 minutes. After completion of the addition, the reaction mixture was allowed to attain 25-30° C. and stirred at this temperature for 2 hours. After completion of the reaction, the reaction mixture was poured into a mixture of conc. HCl (0.4vol) and water (15vol) and stirred for 15 minutes. The reaction mixture was extracted with ethyl acetate and the combined organic extracts were washed with water. The organic layer was dried over sodium sulfate and concentrated under reduced pressure to give the product as yellow oil. Molar yield=96%; Enantiomeric excess=99%.

### (S) 3-Nitromethyl-5-methyl-hexanoic acid

(S) Ethyl 5-methyl-3-nitromethyl-hexanoate (1eq) was dissolved in THF-water (10vol, 2:1), lithium hydroxide (2.5eq) was added and the reaction mixture stirred for 3-4 hours. The reaction was monitored by TLC. At the end of the reaction, the reaction mass was concentrated to remove THF at 35° C. under reduced pressure. Water (5vol) was added to the aqueous mass and extracted with ethyl acetate, acidified with conc. HCl (1vol) and extracted with DCM. The combined DCM layer was washed with water (10vol). Concentration under reduced pressure at 35-40° C. afforded the product as an oil. Molar yield=85%; Enantiomeric excess>99%.

### Pregabalin (2)

Hydrogen was bubbled through a solution of (S) 3-nitromethyl-5-methyl-hexanoic acid (1eq) in methanol (15vol) in the presence of 60% (w/w) of wet 5% palladium on carbon. After completion of the reaction (5-8 hours), the reaction mixture was filtered through a Celite® bed and the filtrate concentrated under reduced pressure to give pregabalin as an oil/sticky solid. The crude product was crystallized from hot 2-propanol/water 1:1 (10vol) to obtain the product as white solid. Molar yield=35%; Enantiomeric excess>99%; HPLC purity=99.6%.

¹H NMR spectrum (D₂O+1 drop of DCl) ppm: 2.87 (d, J=6.3 Hz, 2H); 2.34 (m, 2H); 2.08 (m, 1H); 1.48 (m, 1H); 1.08 (t, J=7.2 Hz, 2H); 0.73 (d, J=6.6 Hz, 3H); 0.71 (d, J=6.6 Hz, 3H).

Mass Spec (electro spray ionization): (M+H)⁺ 160.2; (M−H₂O+H)⁺ 142.2.

Theoretical Preparation of (R) ethyl 5-methyl-3-trifluoromethanesulfonyl-hexanoate

Pyridine (5eq) is added to a solution of (R) ethyl 5-methyl-3-hydroxy-hexanoate (1eq) in DCM (10vol) under N₂ at −78° C. Tf₂O (2eq) is then added dropwise and the mixture is stirred at −78° C. for a further 20 minutes, before warming to 0° C. and stirring for a further 2-3 hours. The reaction is monitored by TLC. After completion of the reaction, the mixture is diluted with DCM, washed with 0.1M HCl then with water. The organic fraction is dried over MgSO₄, filtered, and the solvent removed under vacuum to give the crude product. Column chromatography of the crude product using hexane/ethyl acetate yields the product.

Theoretical Preparation of (S) ethyl 5-methyl-3-nitromethyl-hexanoate

DBU (1.05eq) is added dropwise over 30 minutes to a solution of (R) ethyl 5-methyl-3-trifluoromethanesulfonyl-hexanoate (1eq) in nitromethane (4vol) at 0-5° C. After completion of the addition, the reaction mixture is allowed to attain 25-30° C. and the mixture is stirred at this temperature for 2 hours. After completion of the reaction, the reaction mixture is poured into a mixture of conc. HCl (0.4vol) and water (15vol) and stirred for 15 minutes. The reaction mixture is extracted with ethyl acetate and the combined organic extracts are washed with water. The organic layer is dried over sodium sulphate and concentrated under reduced pressure to give the product.

What is claimed is:

1. A process comprising one or more steps selected from:
(a) the reaction of 4-methyl-2-pentanone (I) with the compound X-G to give the keto intermediate (II): 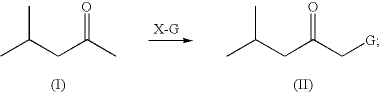
and/or
(b) the reduction of the keto intermediate (II) to the hydroxy intermediate (III): 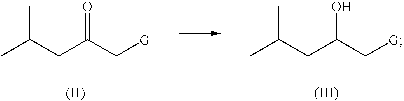
and/or
(c) the displacement of the hydroxyl group of intermediate (III) by a group Y to give intermediate (IV), or the activation of the hydroxyl group of intermediate (III) to give intermediate (V): 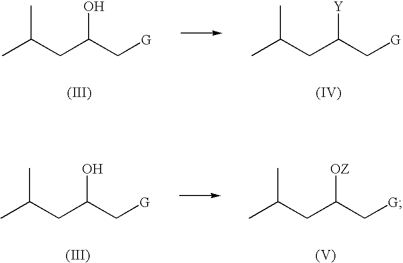
and/or
(d) the reaction of intermediate (IV) or (V) with nitromethane in the presence of a base to give the nitro-derivative (VI): 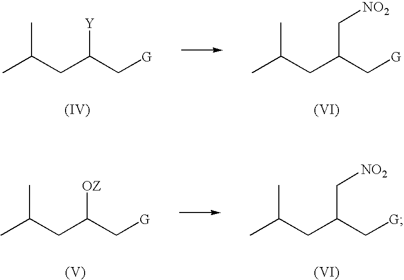
wherein:
X is a suitable leaving group such as a halo, alkoxy, —O-acyl, thio or sulfonate group,
G is a carboxylic acid group or a functional group that is readily converted into a carboxylic acid group,
Y is a suitable leaving group such as a halo group, and
Z is any group that is capable of enhancing the capacity of a hydroxyl group as a leaving group, such as an acyl or sulfonyl group.

2. A process according to claim 1, comprising:
(i) the reduction of the keto intermediate (II) to the hydroxy intermediate (III); and/or
(ii) an asymmetric reduction of the keto intermediate (II) to the hydroxy intermediate (III).

3. A process according to claim 1, for the preparation of racemic pregabalin (1) or (S)-(+)-3-aminomethyl-5-methyl-hexanoic acid (2):

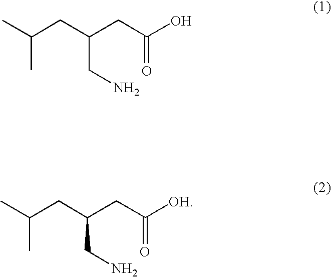

4. A process according to claim 1, wherein:
(i) the group G is chiral; and/or
(ii) the group G is a carboxylic ester, a nitrile, a phenyl, an oxazine, an optionally protected aldehyde or ketone, an alkene, an oxazole, an oxazoline, an ortho-ester, a borane or diborane, a nitro, a hydroxy or an alkoxy group; and/or
(iii) the group G is a carboxylic ester group represented by the formula —CO₂R¹, wherein R¹ is selected from an optionally substituted alkyl, alkenyl, alkynyl, aryl, arylalkyl, arylalkenyl, arylalkynyl or silyl group; and/or
(iv) X is selected from a halo group, or an optionally substituted alkoxy or —O-acyl group; and/or
(v) X is —OR¹, wherein R¹ is selected from an optionally substituted alkyl, alkenyl, alkynyl, aryl, arylalkyl, arylalkenyl, arylalkynyl or silyl group; and/or
(vi) Y is selected from —Cl, —Br or —I; and/or
(vii) Z is selected from a —SO₂R², —SO₂OR², —NO₂, —COR², —P(═O)(OR²)₂ or —B(OR²)₂ group, wherein each R² is independently selected from hydrogen, a halogen, or an optionally substituted alkyl, alkenyl, alkynyl, aryl, arylalkyl, arylalkenyl or arylalkynyl group, and wherein any two R² groups may together with the atoms to which they are attached form a ring; and/or
(viii) Z is selected from a —SO₂R² or —SO₂OR² group, wherein R² is independently selected from hydrogen, a halogen, or an optionally substituted alkyl, alkenyl, alkynyl, aryl, arylalkyl, arylalkenyl or arylalkynyl group; and/or
(ix) Z is selected from a —SO₂R² or —SO₂OR² group, wherein R² is independently selected from a halogen, or an alkyl, aryl or arylalkyl group optionally substituted with one or more groups selected from —F, —Cl, —Br or —NO₂; and/or
(x) —OZ is selected from a tosylate, brosylate, nosylate, mesylate, tresylate, nonaflate or triflate group.

5. A process according to claim 4, wherein R¹ is:
(i) an optionally substituted alkyl or arylalkyl group; and/or
(ii) a methyl, ethyl or benzyl group; and/or
(iii) an ethyl group; and/or
(iv) chiral.

6. A process according to claim 1, wherein in step (a):
(i) 4-methyl-2-pentanone (I) is reacted with the compound X-G in the presence of a base; and/or
(ii) 4-methyl-2-pentanone (I) is reacted with the compound X-G in the presence of sodium hydride.

7. A process according to claim 1, wherein in step (b):
(i) the keto compound (II) is reduced to the hydroxy compound (III) with a reducing agent selected from a borohydride, a cyanoborohydride, diborane or another hydride reducing agent; and/or
(ii) the keto compound (II) is reduced to the hydroxy compound (III) with sodium borohydride; and/or
(iii) the reduction involves an asymmetric reduction of keto intermediate (II) to hydroxy intermediate (III); and/or
(iv) the reduction involves an asymmetric reduction of keto intermediate (II) to hydroxy intermediate (IIIa) or (IIIb): 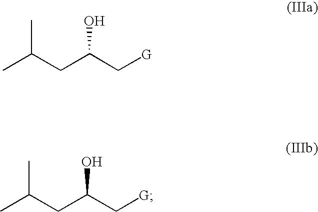
and/or
(v) the reduction involves an asymmetric reduction achieved using an enzyme; and/or
(vi) the reduction involves an asymmetric reduction achieved using Baker's yeast; and/or
(vii) the reduction involves an asymmetric reduction achieved using Baker's yeast of the type Mauri; and/or
(viii) the reduction involves an asymmetric reduction achieved using catalytic hydrogenation; and/or
(ix) the reduction involves an asymmetric reduction achieved using catalytic hydrogenation, wherein the catalyst is a ruthenium complex; and/or
(x) the reduction involves an asymmetric reduction achieved using catalytic hydrogenation, wherein the catalyst is [(S)Ru(BINAP)Cl₂]₂:NEt₃.

8. A process according to claim 7, further comprising:
the separation of hydroxy intermediate (Ma) from hydroxy intermediate (IIIb); and/or
(ii) the separation of hydroxy intermediate (IIIc) from hydroxy intermediate (IIIb), wherein the separation is the separation of enantiomers; and/or
(iii) the separation of hydroxy intermediate (IIIa) from hydroxy intermediate (IIIb), wherein G is chiral and the separation is the separation of diastereoisomers.

9. A process according to claim 1, wherein in step (c):
(i) intermediate (IV) is generated from intermediate (III) via an S_{N}2 displacement of an activated hydroxyl group by Y⁻; and/or
(ii) intermediate (IV) is generated from intermediate (III) via an S_{N}2 displacement of an activated hydroxyl group by Y⁻, wherein the hydroxyl group is activated in-situ; and/or
(iii) Y is a halogen and intermediate (IV) is generated from intermediate (III) using Y₂ and R^{x}₃P, or using HY, PY₃, PY₅, an N-halosuccinimide or SOY₂, wherein each R^{x} is independently selected from an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton; and/or
(iv) Y is a halogen and intermediate (IV) is generated from intermediate (III) using an azidodicarboxylate, an alkyl halide and R^{x}₃P, wherein each R^{x} is independently selected from an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton; and/or
(v) intermediate (IVa) is generated from intermediate (IIIa): 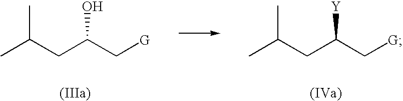
and/or
(vi) intermediate (V) is generated from intermediate (III); and/or
(vii) intermediate (Va) is generated from intermediate (IIIb):

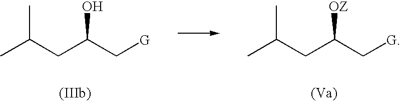

10. A process according to claim 1, wherein in step (d):
(i) the base used is an organic base such as an alkali metal alkoxide, or a tertiary amine such as DBU, triethylamine, N,N-diisopropyl ethyl amine, DBN, or DMAP, or an inorganic base such as an alkali metal carbonate or an alkali metal hydroxide; and/or
(ii) the base used is DBU; and/or
(iii) the nitro-derivative (VIa) is generated from intermediate (IVa): 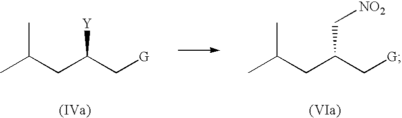
and/or
(iv) the nitro-derivative (VIa) is generated from intermediate (Va):

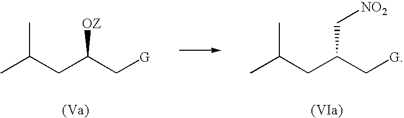

11. A process according to claim 1, further comprising:
(e) the conversion of group G into a carboxylic acid group or a salt thereof and/or
(f) the reduction of the —NO₂ group to a —NH₂ group or a salt thereof.

12. A process according to claim 11, wherein:
(i) the group G is a carboxylic ester group represented by the formula —CO₂R¹, wherein R¹ is selected from an optionally substituted alkyl, alkenyl, alkynyl, aryl, arylalkyl, arylalkenyl, arylalkynyl or silyl group, and wherein the carboxylic acid group or a salt thereof is generated by hydrolysis; and/or
(ii) the group G is a carboxylic ester group represented by the formula —CO₂R¹, wherein R¹ is selected from an optionally substituted alkyl, alkenyl, alkynyl, aryl, arylalkyl, arylalkenyl, arylalkynyl or silyl group, and wherein the carboxylic acid group or a salt thereof is generated by hydrolysis using LiOH; and/or
(iii) step (f) is performed after step (e); and/or
(iv) the reduction of the —NO₂ group to a —NH₂ group is performed using catalytic hydrogenation; and/or
(v) the reduction of the —NO₂ group to a —NH₂ group is performed using catalytic hydrogenation, wherein the catalyst is Pd/C.

13. A process for the preparation of (S)-(+)-3-aminomethyl-5-methyl-hexanoic acid (2), comprising resolution of racemic pregabalin (1) prepared by a process according to claim 3.

14. A compound selected from: 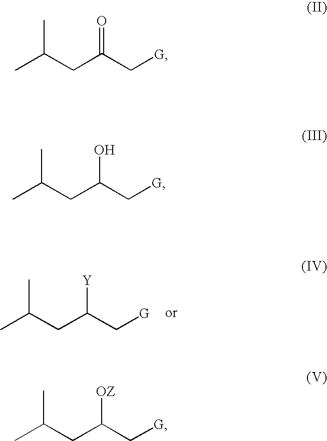
or a salt, tautomer, or stereoisomer thereof, wherein:
G is a carboxylic acid group or a functional group that is readily converted into a carboxylic acid group,
Y is a suitable leaving group such as a halo group, and
Z is any group that is capable of enhancing the capacity of a hydroxyl group as a leaving group, such as an acyl or sulfonyl group.

15. (S)-(+)-3-aminomethyl-5-methyl-hexanoic acid prepared by a process according to claim 1.

16. Enantiomerically pure (S)-(+)-3-aminomethyl-5-methyl-hexanoic acid.

17. Enantiomerically pure (S)-(+)-3-aminomethyl-5-methyl-hexanoic acid, prepared by a process according to claim 1.

18. A pharmaceutical composition comprising the (S)-(+)-3-aminomethyl-5-methyl-hexanoic acid according to claim 15.

19. A pharmaceutical composition comprising the (S)-(+)-3-aminomethyl-5-methyl-hexanoic acid according to claim 16.

20. A pharmaceutical composition comprising the (S)-(+)-3-aminomethyl-5-methyl-hexanoic acid according to claim 17.

21. A method of treating or preventing epilepsy, pain, neuropathic pain, cerebral ischaemia, depression, psychoses, fibromyalgia or anxiety, comprising administering to a patient in need thereof a therapeutically or prophylactically effective amount of (S)-(+)-3-aminomethyl-5-methyl-hexanoic acid according to claim 15.

22. A method of treating or preventing epilepsy, pain, neuropathic pain, cerebral ischaemia, depression, psychoses, fibromyalgia or anxiety, comprising administering to a patient in need thereof a therapeutically or prophylactically effective amount of (S)-(+)-3-aminomethyl-5-methyl-hexanoic acid according to claim 16.

23. A method of treating or preventing epilepsy, pain, neuropathic pain, cerebral ischaemia, depression, psychoses, fibromyalgia or anxiety, comprising administering to a patient in need thereof a therapeutically or prophylactically effective amount of (S)-(+)-3-aminomethyl-5-methyl-hexanoic acid according to claim 17.
